(19) United States
(12) Patent Application Publication
(10) Pub. No.: US 2012/0282634 A1
(43) Pub. Date: Nov. 8, 2012

(54) BLOOD SEPARATION SYSTEM AND METHOD FOR A DRY TEST STRIP

(75) Inventors: Gary Hughes, Anderson, IN (US); Aniruddha Patwardhan, Indianapolis, IN (US); Darby Mcchesney, Lawrenceville, NJ (US); Robert Harper, Marlton, NJ (US); Heather Tomeo, Avon, IN (US); Frank LaDuca, East Brunswick, NJ (US)

(73) Assignee: Polymer Technology Systems, Inc., Indianapolis, IN (US)

(21) Appl. No.: 13/453,465

(22) Filed: Apr. 23, 2012

Related U.S. Application Data
(60) Provisional application No. 61/478,392, filed on Apr. 22, 2011.
(60) Provisional application No. 61/479,280, filed on Apr. 26, 2011.

(51) Int. Cl. G01N 33/566

(52) U.S. Cl. 435/7.25

(57) ABSTRACT

A dry test strip layer for filtering red blood cells includes a Borosilicate Glass Fiber layer and lectin, impregnated in the borosilicate layer, such that the dry test strip is configured to filter red blood cells from a blood sample.

### PRIORITY

This application claims the benefit of U.S. Provisional Application No. 61/478,392 filed Apr. 22, 2011, and U.S. Provisional Application No. 61/479,280 filed Apr. 26, 2011. The entirety of both disclosures is hereby incorporated by reference.

### FIELD OF THE INVENTION

The Blood Separation System And Method For A Dry Test Strip in general relates to bodily fluid analysis systems including a disposable test strip, with particular application to on-site testing of particular analytes in blood. The Blood Separation System And Method For A Dry Test Strip provides for an improved methodology for the separation of blood.

### BACKGROUND OF THE INVENTION

The Blood Separation System And Method For A Dry Test Strip in general relates to bodily fluid analysis systems including a disposable test strip and a spectrophotometric sensing device, with particular application to on-site testing of particular analytes in blood.

The level of certain analytes in blood and other body fluids is often used to diagnose disease, determine disease risk factors, monitor the course of a therapy, or determine the presence of illicit drugs. For example, analytes carried in blood have been evaluated to determine various cholesterol and triglyceride levels as a significant indicator of risk of coronary heart disease. Many assays require that the red blood cells be removed from the sample prior to the measurement of the test strip. This is because the presence of red blood cells can affect optical measurements of color produced to indicate the amount of an analyte in the sample.

A dry test strip assembly includes a dry test strip carrier and a fluid permeable strip. The dry test strip carrier generally is made of a plastic having a tensile strength of about 4,800 pounds per square inch (psi). The permeable strip includes several layers of material to separate the blood components, react the blood plasma with a particular reagent or reagents, and obtain a signal indicative of the concentration of the analyte. All of the above systems depend on the flow of the bodily fluid, i.e., blood, through the system as the driving force to separate the unwanted components from the components to be tested. A rectangular test membrane that is significantly larger than the area of the circular opening through which a spectrophotometer reads the strip enhances this feature to encourage flow and prevent blood pooling in the test area of the membrane. These flow properties are determined by the permeable materials from which the strip material is made and by the carrier for the strip. If the strip is held loosely in the carrier, flow is augmented, but the strip can move, which can lead to erroneous results. If the strip is held firmly, damage can result which leads to erratic results as well as inaccuracies. Thus, test strip carriers have been designed that permit vertical and lateral flow through most of the strip but tightly hold other parts of the strip. The design of dry test strips and carriers also is constrained by the need to manufacture the strip. The strip and carrier should be able to be manufactured and assembled quickly but without negatively affecting the reliability and accuracy of the strip.

Before testing for blood analytes or other substances can be conducted, red blood cells must be removed from the blood sample. This is especially true in the case of those detection methods that involve a color change of the sample since the presence of red blood may significantly affect the sample color. At the same time, such layers are necessarily durable enough to withstand the pressure applied by a test strip holder.

### SUMMARY

The improved methodology utilizes lectins in the blood separation layer. Therefore, an improved test strip includes a plurality of layers, one of the layers being a blood separation layer, the separation layer including lectin. In one embodiment, the lectin is from kidney beans. In one embodiment, the blood separation layer includes Crude Phaseolus Vulgaris Lectin PHA-P. Optionally, it also includes Poly Vinyl Alcohol. Optionally, the layer also includes sodium salt. Optionally, the layer also includes D-(+)-Trehalose dehydrate. Optionally, the layer also includes Neo Protein Saver. Optionally, the layer is made out of Borosilicate Glass Fiber. Optionally, this glass fiber layer is D-23. The proposed combination provides a novel blood filtering layer.

In one embodiment, a method of determining a characteristic of an analyte from a plurality of analytes in a bodily fluid includes providing the bodily fluid containing the analyte and one or more non-selected analytes. The method further includes providing a dry test strip having a well with porous layers within the well that allow the plurality of analytes to pass, creating a vertical column of the analytes having a defined volume. The method further includes applying the bodily fluid to the well in the dry test strip. The method further includes reacting the analyte in the bodily fluid with a reactant in the dry test strip to provide an indication of the characteristic while preventing the one or more non-selected analytes from participating in the reaction, wherein the dry test strip includes a blood separation layer having lectins. Optionally, the blood separation layer is non-brittle and can withstand the pressure of the test strip. In one alternative, the blood separation layer is made out of Borosilicate Glass Fiber. Optionally, the blood separation layer is impregnated with Poly Vinyl Alcohol.

In one embodiment, a system for determining a characteristic of a bodily fluid, the system being portable and of a size that can be easily held in a human hand, includes a test strip having a test area and containing a reagent capable of interacting with the bodily fluid to determine the characteristic, wherein the test strip contains layers that slow a flow of red blood cells in the bodily fluid; and the test strip further includes a red blood cell filtering layer, wherein the red blood cell filtering layer includes lectins. The system further includes a test strip holder. The test strip holder includes a test holder base having a test strip support supporting the test strip, a sensor port communicating with the test strip, a test holder cap having a sample port, and a projecting flange. The test holder base and the cap include an engagement mechanism, the cap secured to the test holder base with the test strip held between the flange and the test strip support along essentially the entire periphery of the test area, wherein the flange is of a length such that the distance between the test strip support and the distal end of the flange is less than the uncompressed thickness of the test strip causing the distal end of the flange and the test strip support to pinch the test strip. Optionally, the red blood cell filtering layer is non-brittle and can withstand the pressure of the test strip. Alternatively, the red blood cell filtering layer is made out of Borosilicate Glass Fiber. Optionally, the blood filtering layer is impregnated with Poly Vinyl Alcohol.

In one embodiment, a carrier system for a diagnostic dry test strip for use in measuring an analyte in a fluid sample includes a carrier base including a test strip well adapted for receiving a dry test strip and a test port communicating with the well and enabling the test strip to be observed, wherein the dry test strip includes a red blood cell separation layer having lectins. The carrier system further includes a cover having a sample opening and engagement elements on the carrier base, the cover configured to engage the cover to the carrier body with the sample opening aligned over the test port, and the dry test strip compressed between the carrier base and the cover. The engagement elements include a maximum dry test strip compression stop controlling the maximum compression on the dry test strip and a minimum dry test strip compression stop controlling the minimum compression on the dry test strip. Optionally, the red blood cell separation layer is non-brittle and can withstand the pressure of the test strip. Alternatively, the red blood cell separation layer is impregnated with Poly Vinyl Alcohol.

In one embodiment, a dry test strip layer for filtering red blood cells includes a Borosilicate Glass Fiber layer and lectin, impregnated in the borosilicate layer, such that the dry test strip is configured to filter red blood cells from a blood sample.

In one embodiment, a method of converting a hydrophobic filtering layer for use in red blood cell separation includes providing a filtering layer and adding lectins causing the filtering layer to become more hydrophilic. Optionally, the filtering layer is a borosilicate glass layer. In one alternative, the method further includes adding poly vinyl alcohol causing the filtering layer to be less brittle and improving its hydrophilic nature. In another alternative, the method further includes adding either Sorbitol/Mannitol or Sucralose/Sorbitol.

### BRIEF DESCRIPTION OF THE DRAWINGS

FIG. 1 is a perspective view of an embodiment of a Blood Separation System And Method For A Dry Test Strip;

FIG. 2 is an exploded perspective view of another exemplary embodiment of a Blood Separation System And Method For A Dry Test Strip;

FIG. 3 is an exploded perspective view of another exemplary embodiment of a Blood Separation System And Method For A Dry Test Strip;

FIG. 4 is an exploded perspective view of a Blood Separation System And Method For A Dry Test Strip;

FIG. 5 is a bottom plan view of the cap portion of the test strip assembly of FIG. 4;

FIG. 6 is a cross-sectional view of the cap of FIG. 5 taken through the line 18-18 of FIG. 5; and

FIG. 7 is a cross-sectional view of the assembled test strip assembly of FIG. 4.

### DETAILED DESCRIPTION OF THE PREFERRED EMBODIMENT

For the purposes of promoting an understanding of the principles of the Blood Separation System And Method For A Dry Test Strip, reference will now be made to the embodiments illustrated in the drawings and described in the following written specification. It is understood that no limitation to the scope of the invention is thereby intended. It is further understood that the Blood Separation System And Method For A Dry Test Strip includes any alterations and modifications to the illustrated embodiments and includes further applications of the principles of the invention as would normally occur to one skilled in the art to which it pertains. It should also be understood that, in accordance with the patent law, the drawings are not intended to be precise engineering drawings of the invention, but rather are only intended to illustrate the Blood Separation System And Method For A Dry Test Strip. For example, the scale of the drawings and relative size of the various parts are generally altered so as to better illustrate the Blood Separation System And Method For A Dry Test Strip within the constraints of a written document such as this.

An embodiment provided in this disclosure provides for a new blood separation layer. This layer offers increased performance of the assay as compared to previous separation layer methodologies. Embodiments of the Blood Separation System And Method For A Dry Test Strip provide increased performance in a test strip. In one embodiment, the separation layer includes lectin as described below. Furthermore, various configurations of a holder are shown.

The dry test strip assemblies are designed for flow of the bodily fluid through the assembly under the force of gravity. The direction of flow is designated as the vertical direction herein, and the two directions perpendicular to the flow are designated as the horizontal directions herein.

In one embodiment, a blood separation layer is a membrane composed of D-23 blood separation material. The following table describes the composition of chemicals in this layer. The impregnated membrane may be stored normal ambient indoor room temperature. Optimum operating temperatures for the impregnated membrane range from 64 degrees to 86 degrees. The D-23 layer is usually used for horizontal flow separation. Typically, those skilled in the art would not use this layer for red blood cell separation due to its highly hydrophobic nature. Typically, D-23 would be used as a filter for liquid or air. In this case it has been converted to a vertical flow layer. In order to accomplish this transformation, the layer must be rendered hydrophilic by using PVA. The ranges of PVA that provide for a layer that is hydrophilic but yet not overly brittle range from 0.1 to 0.5 percent weight by volume. This layer is a glass fiber layer. In order to capture red blood cells, but not interfere with the transmission of cholestrol cells, which in many combinations utilizing the blood separation layer are configured to measure, Phaseolus Vulgaris Lectin is added to the separation layer. The resulting layer is quite brittle. Poly Vinyl Alcohol was added, resulting in a layer that filters red blood cells, does not negatively affect the flow of cholesterol due to the complementary hydrophobic nature of the lectins, and can withstand the pressure of compression and use exerted on test strip layers.

TABLE 1

| Item Description | Qty | Unit | Vendor |
|---|---|---|---|
| D.I. Water (distilled | 700.00 | mL | N/A |
| water) | | | |
| Poly Vinyl Alcohol (30-50k) | 1.000 | gm | Sigma |
| Sodium Chloride | 8.728 | gm | Sigma |
| PIPES, Sequisodium | 3.724 | gm | Sigma |
| salt | | | |
| pH 6.5 | | | |
| D-(+)-Trehalose | 3.600 | gm | Sigma |
| dihydrate | | | |
| Neo Protein Saver | 0.720 | gm | Toyobo |
| Crude Phaseolus | 0.500 | gm | Sigma |
| Vulgaris | | | |
| Lectin PHA-P | | | |
| Q.S. with D.I. Water to | 1000.00 | mL | N/A |
| D-23 Borosilicate | 2400.00 | In² | IW |
| Glass Fiber, | | | Tremont |
| 6″ wide | | | |

The above Table 1 describes an example of an embodiment of a blood separation layer. Within the parameters of the example of above, the distilled water amount used may vary according to test strip size and various drying times desired.

Embodiments include the use of PHA-P lectins (Phaseolus Vulgaris Lectin) to retard Red Blood Cell flow. Concentration of PHA-P was originally at 100 mg/100 ml of solution and later was reduced to 50 mg/100 ml because D-23 slowed down the plasma flow. Applicants note that D-23 has never been used as a red blood cell separation layer. D-23 is extremely hydrophobic and therefore a sample would not easily penetrate the D-23. In order to facilitate the flow of the sample it was necessary to make D-23 layer more hydrophilic. Applicants has tested numerous other red blood cell separation layers for use in an analyte strip, however, none provide the performance of the D-23 layer as enhanced by the chemistries described herein. Separation layers tried include: Fusion 5 by Whatman, VF2 by Whatman, Ahlstrom Grade 142, and Ahlstrom Grade 144. Other Borosilicate Glass Fiber layer with similar characteristics will perform similarly as described herein when treated accordingly.

In order to improve the hydrophilic property of D-23, poly vinyl alcohol (PVA) was also used. Too much PVA will clog the glass fibers not allowing the lipoprotein to react. For a Fusion 5 layer, the optimum amount was between 0.1% and 0.5%. For D-23, it seems that the slope does not start to bend over until after 1% PVA. If no PVA is added, in actual cholestrol assays, the quantification of % R yields erratic endpoints.

Additionally, flow may be improved certain additives. The addition of Sorbitol/Mannitol and Sucralose/Sorbitol may increase flow. Preferred combinations of Sorbitol/Mannitol include a 1 to 1 ratio, from 1.5-5.5% weight by volume and even more preferably 3.5%. Preferred combinations of Sucralose/Sorbitol include a 1 to 1 ratio, from 1-5% weight by volume and even more preferably 3%. Sorbitol/Mannitol may be preferred over Sucralose/Sorbitol since it is less likely to interfere with cholestrol readings, but in alternative types of tests Sucralose/Sorbitol may be preferred.

The viscosity of the whole blood sample determines the timing when the plasma reaches the reaction layer. For instance, a sample with high triglycerides will take longer to reach the reaction membrane than a sample with low triglycerides. Therefore, a sufficient flow must be maintained to provide for a large cross-section of samples to reach the reaction layer and react sufficiently for measurement.

These embodiments of blood separation layers offer increased performance of separation. This layer may be incorporated into any of the examples of layers listed below.

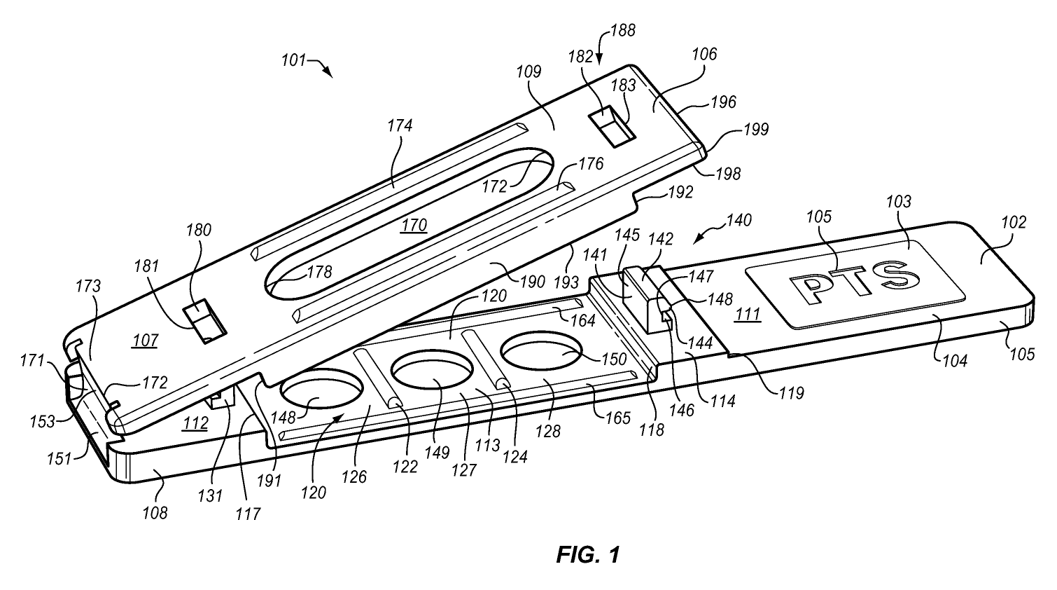

FIG. 1 shows a perspective view of a preferred embodiment of a dry test strip carrier 101 according to the invention in the open position. Carrier 101 has a proximal end 105 and a distal end 108 and includes base 102 and cover 106. Base 102 comprises an elongated plate 104 having a raised back platform 112 and a raised forward platform 114, a depressed area 113 between them, and a tongue portion 111 which is thinner than the platforms 112 and 114. A roughened area 105, in this case in the form of the logo of the assignee, makes it easier to hold the tongue between a thumb and forefinger. Strengthening ribs 164 and 165 are formed along the side of depressed area 113. Ribs 164 and 165 together with the end walls 117 and 118 of depressed area 113 form an enclosed well 120. In this embodiment, three sensor ports 148, 149, and 150 are formed in well 120. Ribs 122 and 124, preferably spaced equidistant between the end ports 148 and 150 and the central port 149, separate well 120 into three separate test wells 126, 127, and 128. Stop members 131 and 141 are formed on platforms 112 and 114, respectively. Stop member 141 includes a pad 144 which determines the minimum distance between the base and cover and a hook-shaped end 148 forming a latch lip 144 which determines the maximum distance between the base and cover. The end 142 of stop member 141 is radiused. Stop member 131 is preferably the mirror image of stop member 141. A hinge member 151 projects from the distal end 153 of base 102. Base end 153 has an indentation 152 at hinge 151, so that when strip assembly 10 is inserted into slot 1214 of reader 1200 (FIG. 12), base plate 104 abuts the end 1220 of slot 1214 rather than hinge 151.

Cover 106 comprises an elongated plate 107 having openings 180 and 182 for receiving stop members 131 and 141, sample port 170, strengthening ribs 174, and compression plate 190 extending away from the bottom surface 198 of cover plate 107. Sample port 170 is preferably an oblong slot with semicircular ends 178 and 179, with a width that is slightly smaller than the diameter of sensor ports 148-150 and a length such that the semicircles of ends 178 and 179 lie inside the radius of the sensor ports 148 and 150. The ends 191 and 192 of compression plate 190 are formed to mesh with the ends 117 and 118 of depression 113 so that compression plate 190 fits snuggly into depression 113. Ribs 164 and 165 are preferably slightly shorter than ribs 122 and 124, so that they do not interfere with the controlled compression of dry test strip 160. A living hinge 171 connects hinge member 151 and end 173 of cover plate 107. In one embodiment, only one end of the hinge 151 is a living hinge, preferably the upper end. In another embodiment, both ends of hinge 151 are living hinges; that is. the hinge is a double hinge. End 173 of cover 106 has an indentation 172 at hinge 171 to prevent the hinge from abutting the end of sample port 170. Proximal end 196 of cover plate 107 is preferably radiused. Stop members 131, 141 are located on base 102 so that the interior walls 137 and 147 of hook-shaped ends 138 and 148 are approximately the same distance apart as the outer edges 181 and 183 of openings 180 and 182.

The structure and chemistry of fluid permeable strip element 160 is known in the art and will not be discussed herein. It may be an LDL or HDL dry test strip element as described in US Patent Application Publication No. 2006/0062688 A1, a glucose dry test strip element, a triglycerides dry test strip element, or a creatinine dry test strip element, though it may be any other dry test strip element known in the art or yet to be developed.

Dry strip assembly 101 is assembled by placing a fluid permeable strip element 160 in well 120 and engaging the engagement elements 140 comprising elements 131, 141, 180, 182, 120, and 190 by pressing cover 106 into base 102 so that stop members 131 and 141 pass through openings 180 and 182, respectively, in cover 106. The radius on the distal ends 132 and 142 cause stop members 131 and 141 to bend slightly as cover 106 is pressed into base 102 until lips 134 and 144 pass through openings 180 and 182, respectively. The stop members 131 and 141 then snap back, and lips 134 and 144 latch over edges 181 and 183, respectively, of openings 180 and 182. Thus, the elements 131, 141. 180, and 182 comprise a snap-on latch 188. The step down from platform 114 to platform 119 provides a small amount of leeway so that if end 199 of cover 106 bends a little under the pressure of closure, it does not interfere with the latching of the stop members 131 and 141.

As cover 106 is closed, compression plate 190 fits into well 120 and compresses dry test strip element 160 against ribs 122 and 124 to form three separate dry test strip test regions that are essentially fluidly isolated from one another. These ribs may be between 0.014 inches and 0.035 inches in height. More preferably, they are between 0.020 inches and 0.030 inches in height. Most preferably, they are 0.025 inches in height. However, the portions of the dry test strip element 160 away from the ribs 122 and 124 are preferably only slightly compressed, or alternatively, uncompressed, so that the carrier does not interfere with the free flow of fluid through the test strip test regions. To apply proper pressure to the dry test strip element, the cover 106 and base 102 preferably are made of a material having a greater tensile strength than prior art carriers, preferably between 10,000 pounds per square inch (psi) and 14,000 psi; more preferably, between 11,000 psi and 13,000 psi; and most preferably, 12,000 psi. The dry test strip may composed of material as described above. Preferably, the distance between the surface of lips 134 and 144 and the bottom of well 120 is controlled to be just slightly smaller than the distance between the top surface 109 of cover plate 107 at edges 181 and 183 and the bottom surface 193 of compression plate 190 plus the thickness of dry test strip element 160. Alternatively, these distances are set to be essentially equal. In addition, the distance between the surface of landing pads 136 and 146 and the bottom of well 120 is controlled to determine the maximum compression of dry test strip element 160 as the cover 106 is closed. That is, this distance is controlled so that dry test strip element 160 cannot be over compressed so as to damage the dry test strip at ribs 122 and 124 or compact the test strip beyond recovery, which would interfere with the flow of fluid through the strip and decrease accuracy. Thus, the lips 134 and 144 act as a minimum dry test strip compression stop, and the landing pads 136 and 146 act as a maximum dry test strip compression stop.

The compression of the dry test strip 160 also preferably is controlled by adding stiffening ribs 174 and 176 to the cover and stiffening ribs 164 and 165 to the base. The stiffer cover and base reduces inaccuracies in compression due to bending of the plates 104 and 107. It also equalizes the compression in the three test strip test areas 185, 186, and 187.

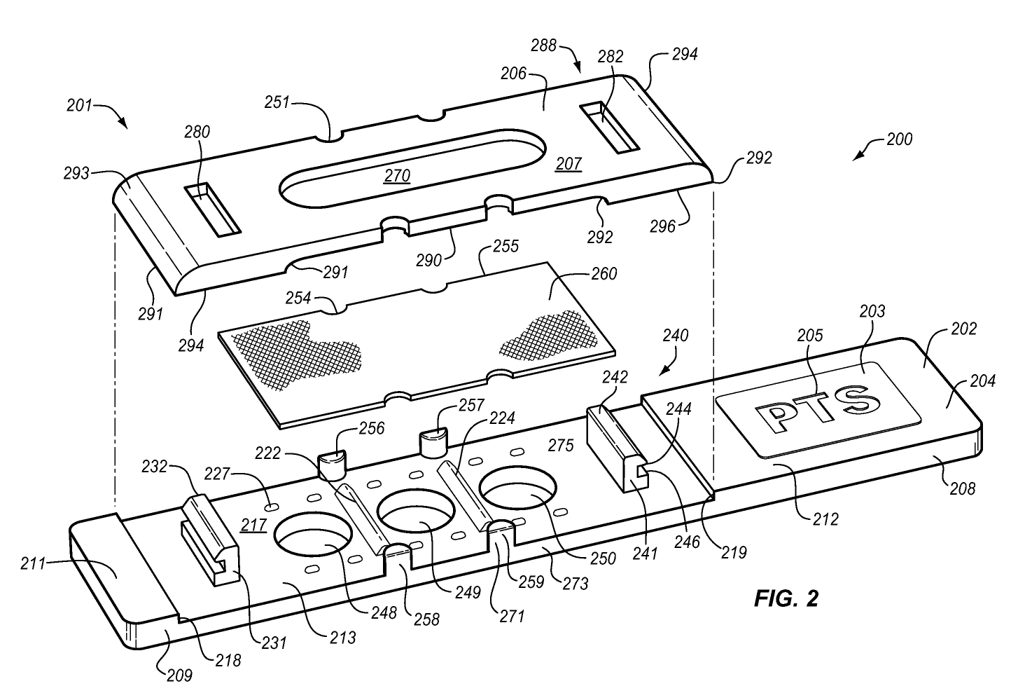

FIG. 2 shows an exploded perspective view of another preferred embodiment of a dry test strip assembly 200 according to the invention having a proximal end 208 and a distal end 209. Assembly 200 includes carrier 201 and dry test strip element 260. Carrier 201 includes base 202 and cover 206. Base 202 comprises an elongated plate 204 with raised platforms 211 and 212 at the ends and a depressed platform 213 between them. A roughened area 205 is formed on thumb plate 205. Stop members 231 and 241, ribs 222 and 224, and sensor ports 248, 249, and 250 are formed on depressed platform 213 and are as described in the embodiment of FIG. 1. Alignment posts 256, 257, 258, and 259 also are formed on base 202 protruding from depressed platform 213 at its sides. Preferably, the posts are semicircular with a flat surface, such as 271, aligned with the outer edge, such as 273, of the base and a semicircular inner surface, such as 272. Small protrusions, such as 227, are formed in the surface 217 of depression 213. These protrusions 227 provide an anti-slip friction surface for dry test strip element 260.

Cover 206 comprises a plate 207 having radiused ends 291 and 292, a recess 290, a sample port 270, openings 280 and 282 for receiving stop members 231 and 241, respectively, and guide grooves, such as 251, which are preferably semicircular. The end walls 291 and 292 of recess 290 are radiused. Recess 290 is essentially the size and shape of dry test strip element 260.

Dry test strip element 260 is as discussed above except that semicircular guide indentations, such as 254, are formed in the longitudinal edges, such as 255.

Dry test strip assembly 200 preferably is assembled by engaging the engagement elements 240. Engagement elements 240 comprise elements 231, 241, 280, 282, and 256-259, while a snap-on latch comprises elements 231, 241, 280, and 282. The elements are engaged by placing dry test strip element 260 on surface 217 of depression 213 with indentations 254 fitting on guide posts 256-258. Cover 206 then is placed over base 202 with guide grooves 251 fitting over guide posts 256-258, dry test strip 260 fitting into recess 290, stop members 232 and 242 snapping into openings 280 and 282, respectively, as described in reference to the embodiment of FIGS. 1-3, and ends 291 and 292 of cover 206 abutting the end walls 218 and 219, respectively, of depression 213. The lower surfaces 294 and 296 of cover 206 rest on surface 217 of depression 213. As in the embodiment of FIG. 1, lips 244 act as a minimum dry test strip compression stop, and the landing pads act as a maximum dry test strip compression stop.

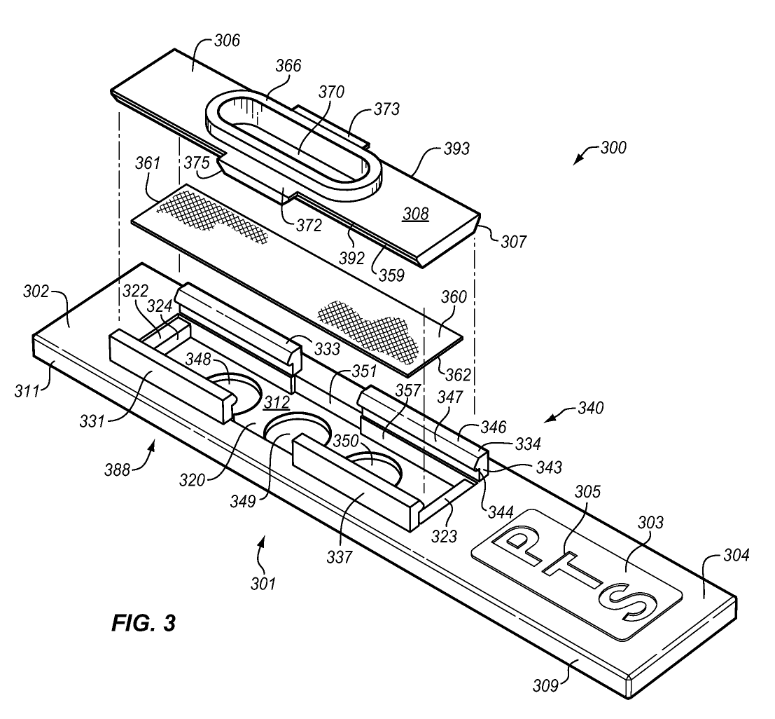

FIG. 3 illustrates another preferred embodiment of a dry test strip assembly 300, this embodiment having a proximal end 309 and a distal end 311. Dry test strip assembly 300 includes test strip carrier 301 and dry test strip element 360. Test strip carrier 301 includes base 302 and cover 306.

Base 302 comprises an elongated plate 304, again with a thumb plate 303 having a roughened area 305. A well 320 is formed in plate 304, and three sensor ports 348, 349, and 350 are formed in the bottom 317 of well 320. Cover landing pads 322 and 323 are formed at the proximal and distal ends of well 320. Rectangular grooves 351 and 352 are formed along the sides 356 and 357 of well 320 in the center portion of the well. Stop members 331, 337, 333, and 334 extend upward from plate 304 aligned with the sides 356 and 357 of well 320. Each stop member, such as 334, includes a vertical pillar, such as 343, at the top of which is a hook-shaped latch member having a ramp 347 and a lip 344. Dry test strip element 360 is as discussed above.

Cover 306 comprises a plate 308 having a sample opening 370. The shape and dimensions of the opening 370 are as described in reference to the openings 170 and 270 of the previous embodiments. However, in the vertical direction, a rib 366 extends vertically from plate 308 and encircles opening 270. Guide members 372 and 373 extend horizontally from the side plate 307, preferably in the center of the elongated length. Each guide member has ramps 375 sloping at an angle to the vertical in a plane perpendicular to the vertical. The longitudinal sides of plate 307 form ramps 359 along the elongated direction.

Dry test strip assembly 301 is assembled by placing dry test strip element 360 in well 320 with its ends 361 and 362 abutting end walls 324 and 325, respectively, of well 320. As will be seen below, dry test strip 360 comprises a multilayered structure including a plurality of membranes. The membranes can either be stacked separately in well 320, or more than one membrane at a time can be placed in well 360. Cover 306 then is placed over base 302 with guide members 372 and 383 aligned over grooves 351 and 352, respectively. Cover 306 then is pressed into base 302 with ramps 375 of guide members 372 and 373 riding on the sides of grooves 351 and 352, ramps 347 of stop members 331-334 riding on ramps 359 of cover plate 306 until the edges 392 and 393 of cover plate 306 snap under lips 344 of stop members 331-334 and the bottom surface 397 of plate 306 at ends 394 and 395 rests on the upper surfaces of cover landing pads 322 and 323. The compression of dry test strip element 360 in well 320 is controlled by cover landing pads 322 and 324 and lips 344, with lips 344 acting as a minimum dry test strip compression stop and the cover landing pads 322 and 323 acting as a maximum dry test strip compression stop. Rib 366 stiffens the cover in the critical central area assisting in controlling compression and equalizing the compression over the three sensor ports 348-350. Similarly, stop members 331-334 stiffen the base 301. In this embodiment, the engagement elements 340 include elements 331, 337, 333, 334, 359, 392, 393, 372, 373, 351, 352, 322, and 323 and the snap-on latch includes elements 331, 337, 333, 334, 359, 392, and 393.

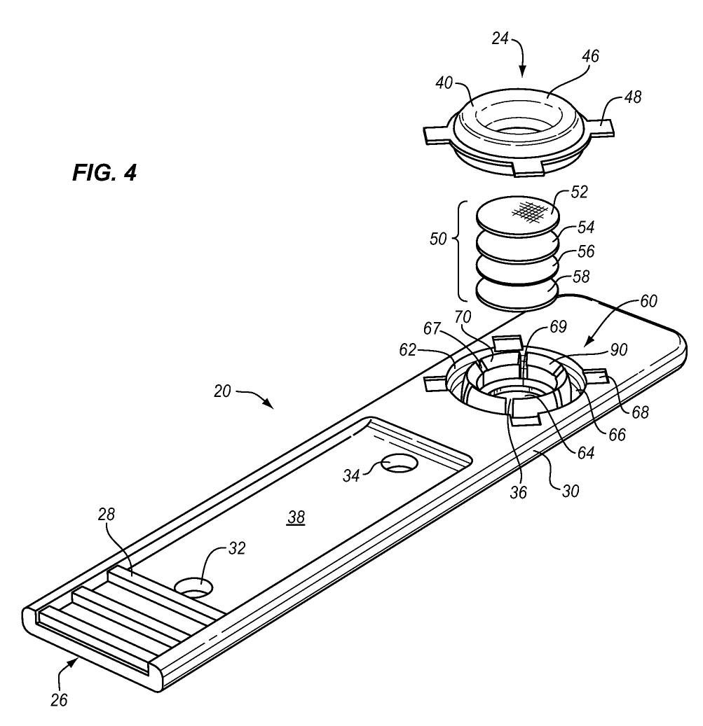

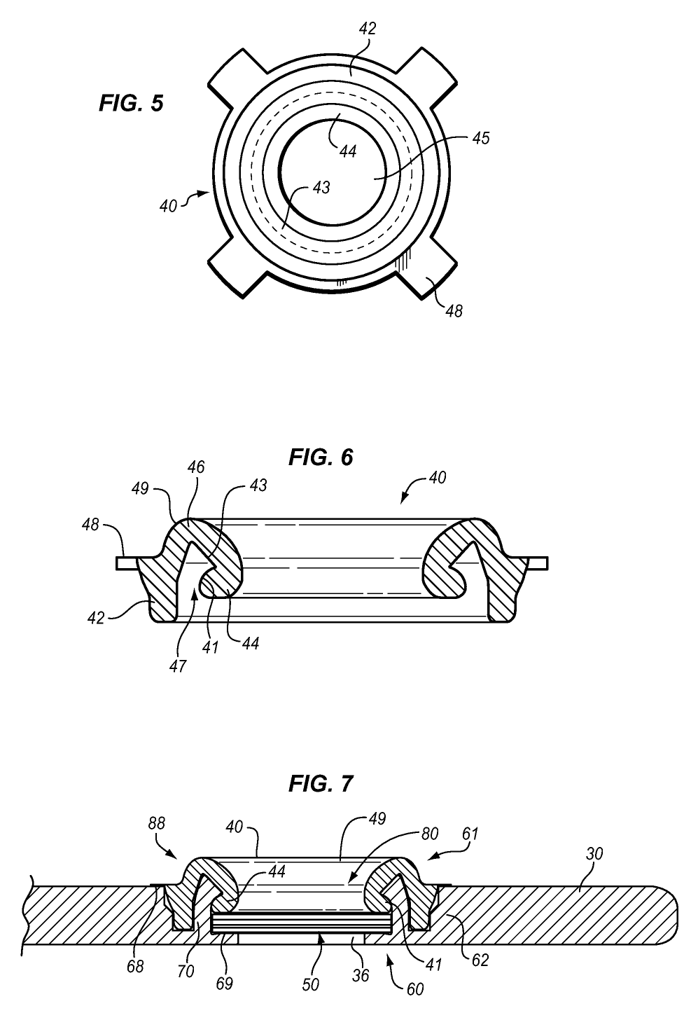

Another exemplary embodiment of a dry test strip assembly 20 according to the invention is shown in FIGS. 4-7. An exploded perspective view of the test assembly 20 is shown in FIG. 4. Test strip assembly 20 includes a preferably elongated test strip carrier body 30, a test strip element 50, and a test strip carrier 24. Test strip carrier 24 includes a carrier base portion 60 and a carrier cap 40. Carrier body 30 includes a grip portion 26, openings 32 and 34, sensor port or test opening 36, and carrier base 60. Grip portion 26 includes raised ribs 28 which permit the fingers to easily grip the carrier body 30.

Preferably, carrier base 60 includes a well 62 formed in body 30, alignment recesses 68, and retainer 90, which preferably is flexible. Well 62 has an upward sloping well wall 83 completely encircling the test opening (sensor port) 36. Retainer 90 preferably comprises fingers 70 and separates well 62 into an inner portion 64 which forms a test strip well 62 and an outer portion 66, which is preferably relatively small in volume, being just big enough to allow fingers 70 to flex. In this disclosure, the term “encircle” does not necessarily mean the encircling structure forms a circle, but rather it has the broader common meaning of “to pass completely around.” In the preferred embodiment, however, the well 62 and fingers 70 do form a circle. In the preferred embodiment, there are four alignment recesses 68 and six fingers 70, though the invention contemplates that any number suitable to perform the functions described below may be used. Each finger 70 includes a stem portion 72, a hook portion 74, and a ramp portion 76 that preferably is formed at an acute angle to a vertical line perpendicular to the plan of body 30. Fingers 70 are separated by channels 67. The bottom of well 62 forms a test strip support 69 around port 36 on which, as will be seen below, the test strip element 50 rests, as best shown in FIG. 9.

FIG. 4 shows a perspective view and FIG. 7 shows a cross-sectional view of the cap 40 in place over the carrier base 60. Cap 40 includes an outer foot 42, an inner flange 44, and a connecting portion 46, which, as will be seen below, forms the brim 49 of a bodily fluid container 80. The outer foot 42 and inner flange 44 have different lengths, with the inner flange being shorter. The difference in lengths is less than the thickness of test strip assembly 50, so that the inner flange 44 and test strip support 69 engage strip 50 sufficiently to secure it in place. Preferably, the difference is sufficient so that flange 44 and test strip support 69 compress strip element 50 between them. The bottom 43 of connecting portion 46 is shaped to form a groove 47 into which fingers 70 fit snuggly. A lip 41 is formed on flange 44 (FIG. 7) which engages hook 74 to latch cap 40 on carrier base 60. The distal end 84 of flange 44 is smooth and rounded so as not to damage test strip element 50.

Test strip 50 is shown in FIGS. 4 and 7 and preferably is formed of a plurality of layers. Each layer performs a specific function as required by each specific test. Generally, there is a “spreading” or “disbursement” layer 52 to ensure even distribution of the whole blood sample; a “separation” layer 54 to obtain a clarified plasma/serum sample; a layer or layers 56 to hold specific test reagents in sequence as needed by each specific assay; and a final “color” or “test reaction” layer 58 to provide a matrix on which a specific color or test reaction will develop for each specific test. The order of the layers can vary. For example, the separation layer may come before or after the reagent layer(s). The details of the test strip layers are described in US Patent Application Publication No. 2006/0062688. Together the layers 54, 56, and 58 shall be referred to as “reagent layers” because they typically include reagents, to distinguish them from disbursement layer 52. The test strip elements 160, 260, and 360 of the previous embodiments are made in a similar fashion, except for the fact that they are differently shaped and, in the regions above the separate test ports, such as in each of the separate fluid tight compartments of the embodiments of FIGS. 1-4, they contain different reagents.

The purpose of the red blood separation layer is to remove blood cells from the analyte liquid and to further add to the reagent/solvent contact time to continue the process of getting the reagent into solution. Preferably, it is made of an asymmetrically porous material; that is, the pore size varies through the material. Preferably, the side with the large pores is up.

A feature of the invention is that each layer of the test strip assembly is engineered to perform specific functions, and at the same time the various layers cooperate so that the test strip assembly as a whole operates to provide more accurate and reliable results. The layers together operate to create a vertical flow of sample liquid essentially across the entire test strip assembly. The red blood cells tend to move slower than the rest of the sample, or get removed from the sample by the lectin blood separation layer and therefore, during the time in which the colorimetric reagent is reacting, will be contained in the layers above the reaction layer and will not be in the reaction layer. However, the other analytes may or may not be in the reaction layer. Since they are rendered non-reactive by the reagents in the reaction layer, whether or not they are present is not of great importance. This feature allows the reaction layer to be much thinner than prior art reaction layers and still yield an accuracy associated with reaction layers that are much thicker.

Another feature of the invention is that the structures of the invention create a sample container, the sidewalls and bottom of which essentially do not pass liquid, and the top of which is open. This creates several advantages that result in a more accurate and reliable measurement. First, it results in a well-defined test volume of sample fluid. When the bodily fluid is added to the container, it flows to the bottom and then stops. Only the bodily fluid in the reagent layer, and the adjacent layers in test strips in which the open pore feature discussed above is used, takes part in the reaction. Moreover, at the time of the reaction, this volume is essentially quiescent. Thus, a defined volume of fluid participates in the reaction. This duplicates much more closely the laboratory type test in which a beaker with a defined volume is used in tests, as compared to prior art test strips in which flow, particularly transverse flow, continued to occur during the test, which flow could depend on many variables and was difficult to quantify. Moreover, the fact that flow stops prevents red blood cells from getting through the layers above the test layers. That is, once flow stops, there is no flow or pressure to move the red blood cells. Thus, the layers above the test layer do not have to be completely impenetrable to red blood cells. All they have to do is slow the red blood cells for awhile until the test volume is filled. This again plays back into the feature that the red blood cells do not completely block the pores but permit ease of fluid flow once the reagent is reconstituted.

In the inventive test, if more bodily fluid than is required for the test is placed in the sample port, the fluid in excess of what is required for the test simply fills up the upper portion of the container and does not affect the test. If the excess is too much even for the container, the excess simply overflows the brim and does not affect the test. Thus, the bodily fluid analysis system according to the invention is much less sensitive to the amount of bodily fluid supplied than prior art systems.

The above feature of the invention, i.e., that the test strip holder provides a sample container, the sidewalls and bottom of which essentially do not pass liquid and, therefore, the test is performed on a well-defined volume of fluid, also increases the accuracy of the test because it provides a definitive end point to the test. As disclosed in U.S. Pat. No. 5,597,532, a pseudo end can be determined from measurements of the reflectance through the sensor port. The pseudo end point is defined as the point on the curve where the change in percent reflectance per unit time becomes smaller than a predetermined amount; that is, the slope of the reflectance versus time curve becomes less than a predetermined slope. However, in the prior art after the pseudo end point, the reflectance continues to drop for a considerable time because the reaction continues.

For this strip holder according to the invention, the percent reflectance versus time curve reaches a minimum and then begins to curve upward. This is because only a well-defined amount of plasma takes part in the reaction; and after that plasma reacts, the color begins to fade as the reactants that produce the color oxidize or otherwise begin to break down, and the slope of the reflectance versus time curve becomes zero. The minimum defines an effective end point that is much easier to measure than a pseudo end point. For example, one can set the electronics to select the effective end point when the value of percent reflectance increases for a predetermined number of measured points, for example, three points each taken a second apart. Generally, one will require more than just one increased value of the percent reflectance to determine the effective end point because random noise and other factors can lead to a single increased value for the curve when the curve is actually still continuing downward. The easier-to-measure minimum contributes to the increased accuracy of the test strip according to the invention.

A related feature of the invention is that the test strip holder provides a controlled region for vertical flow of the bodily fluid sample. These features, alone and in combination, eliminate or sharply limit leaching or lateral flow of the sample as bodily fluid flows vertically through the layers. This degree of control translates to the ability to obtain accurate test readings from a reduced blood sample. Accurate results can be obtained with a sample size of as low as 15-20 mirco liters and 40-50 micro liters with the present invention.

A further feature of the invention is that the test strip assembly, such as 50, preferably does not include any glue, adhesive, or other substance to hold it in place. Such substances can get into the test sample and compromise the test to make it less accurate and reliable.

Another feature of the invention is that the reagents used, particularly those in the blood separate layer, are non-hemolytic. That is, they will not rupture the red blood cells. This prevents the matter from inside the red blood cells from compromising the test. Preferably, the reagents are hypertonic; that is, the reagent in solution has a higher osmotic pressure than the osmotic pressure within the red blood cells. Thus, if there is any flow of water, it will be from within the cell to outside the cell. The reverse could cause the cells to gain water until they rupture. However, the reagents are selected so that the degree of hypertonicity is low. Otherwise, the liquid from within the blood cells could dilute the bodily fluid to be analyzed.

The design methodology of the invention is a self-consistent and self-reinforcing process. The materials and chemical processes of the invention are carefully engineered so that more accurate and more reliable results can be achieved with a smaller amount of reagent and a correspondingly smaller test strip assembly. Because the results that can be achieved are more accurate and can be achieved with a smaller amount of reagent, more flexibility is permitted in the selection of materials in the layers and the reagents. For example, membranes that retain and hold relatively small amounts of liquid can be selected over fabrics that hold large amounts of fluid, while fabrics that hold large amounts of fluid can also be used advantageously where appropriate. The ability to use a wider variety of materials enables the engineer to design a test that is closely akin to a laboratory analysis. That is, laboratory analyses can be very accurate because the order and timing of the reactions can be carefully controlled. One can add an accurately measured amount of a first reactant to an accurately measured amount of solvent, allow a first reaction to occur, then add an accurately measured amount of second reactant, and perform a second reaction, and so on. The ability to use a wide variety of different materials allows one to control the order and timing of the reactions in a similar manner. The first reaction is placed closest to the top in the vertical structure of the test strip assembly. The timing of the second reaction can be controlled by choosing the materials of the first reactant layer and the adjoining layers to control the flow time through the layers, and so on.

The separation layer including lectin is useful in a variety of assays. Now that a dry test strip assay has been disclosed that mimics many of the features of a laboratory assay, such as use of a well-defined test volume, reaction order and timing controls using a variety of materials, and the ability to remove red blood cells from the reaction while still providing the above two features, these features also may be used to test for total cholesterol, triglycerides, and many other analytes. Further, now that the advantages of a non-precipitating dry test strip, asymmetric membranes, and removal of red blood cells from the detection area without filtering that can clog the system have been disclosed, these features also can be advantageously used for testing of other analytes. Further, although the description has disclosed specific exemplary material layers that perform the features of the invention, now that the functions of the layers and the interrelationships of the layers has been described, many other materials can be substituted which will perform the same functions. In addition, while the invention has been disclosed in terms of specific exemplary reactants, many other reactants that perform the same functions and have some or all of the same advantages can be substituted. Again, while the invention has been disclosed in terms of a particular bodily fluid, i.e., blood and blood plasma, many features of the invention will be useful in testing other bodily fluids, such as urine. Thus, the invention should not be limited to these specific structures, layer materials, reactants, and bodily fluids.

While the separation layer and test strip incorporating a separation layer has been illustrated and described in detail in the drawings and foregoing description, the same should be considered as illustrative and not restrictive in character. It is understood that only the preferred embodiments have been presented; and that all changes, modifications, and further applications that come within the spirit of the invention are desired to be protected.

For instance, while the illustrative embodiments only show a single sample application port and a single corresponding sensor port, multiple sample ports and multiple sensor ports are contemplated.

I claim:

1. A dry test strip layer for filtering red blood cells, the dry test strip layer comprising:
a Borosilicate Glass Fiber layer; and
lectin, impregnated in the borosilicate layer, such that the dry test strip is configured to filter red blood cells from a blood sample.

2. The dry test strip layer of claim 1 where the lectin is from kidney beans.

3. The dry test strip layer of claim 1 where the lectin is Crude Phaseolus Vulgaris Lectin PHA-P.

4. The dry test strip layer of claim 1, further comprising:
Poly Vinyl Alcohol, impregnated in the Borosilicate Glass Fiber layer.

5. The dry test strip layer of claim 1, further comprising:
sodium Salt, impregnated in the Borosilicate Glass Fiber layer.

6. The dry test strip layer of claim 1, further comprising:
D-(+)-Trehalose dehydrate impregnated in the Borosilicate Glass Fiber layer.

7. The dry test strip layer of claim 1, further comprising:
Neo Protein Saver, impregnated in the Borosilicate Glass Fiber layer.

8. The dry test strip layer of claim 1 where the Borosilicate Glass Fiber layer is made out of D-23.

9. The dry test strip layer of claim 1 where the dry test strip layer is non-brittle and can withstand the pressure of a test strip holder.

10. A method of determining a characteristic of an analyte from a plurality of analytes in a bodily fluid, said method comprising:
providing said bodily fluid containing the analyte and one or more non-selected analytes;
providing a dry test strip having a well with porous layers within said well that allow said plurality of analytes to pass creating a vertical column of said analytes having a defined volume;
applying said bodily fluid to said well in said dry test strip; and
reacting the analyte in the bodily fluid with a reactant in said dry test strip to provide an indication of said characteristic while preventing said one or more non-selected analytes from participating in said reaction, wherein the dry test strip includes a blood separation layer having lectins.

11. The method of claim 10 where the blood separation layer is non-brittle and can withstand the pressure of the test strip.

12. The method of claim 10 where the blood separation layer is made out of Borosilicate Glass Fiber.

13. The method of claim 10 where the blood separation layer is impregnated with Poly Vinyl Alcohol.

14. A system for determining a characteristic of a bodily fluid, said system being portable and of a size that can be easily held in a human hand, said system comprising:
a test strip having a test area and containing a reagent capable of interacting with said bodily fluid to determine said characteristic, wherein the test strip contains layers that slow a flow of red blood cells in said bodily fluid and the test strip further includes a red blood cell filtering layer, wherein the red blood cell filtering layer includes lectins;
a test strip holder comprising:
a test holder base having a test strip support supporting said test strip and a sensor port communicating with said test strip, and
a test holder cap having a sample port and a projecting flange;
said test holder base and said cap including an engagement mechanism, said cap secured to said test holder base with said test strip held between said flange and said test strip support along essentially the entire periphery of said test area, wherein said flange is of a length such that the distance between said test strip support and the distal end of said flange is less than the uncompressed thickness of said test strip causing the distal end of said flange and said test strip support to pinch said test strip.

15. The system of claim 14 where the red blood cell filtering layer is non-brittle and can withstand the pressure of the test strip.

16. The system of claim 14 where the red blood cell filtering layer is made out of Borosilicate Glass Fiber.

17. The system of claim 14 where the blood filtering layer is impregnated with Poly Vinyl Alcohol.

18. A carrier system for a diagnostic dry test strip for use in measuring an analyte in a fluid sample, said carrier system comprising:
a carrier base including a test strip well adapted for receiving a dry test strip and a test port communicating with said well and enabling said test strip to be observed, wherein the dry test strip includes a red blood cell separation layer having lectins;
a cover having a sample opening; and
engagement elements on said carrier base and said cover configured to engage said cover to said carrier body with said sample opening aligned over said test port and said dry test strip compressed between said carrier base and said cover;
wherein said engagement elements include: a maximum dry test strip compression stop controlling the maximum compression on said dry test strip, and a minimum dry test strip compression stop controlling the minimum compression on said dry test strip.

19. The carrier system of claim 18 where the red blood cell separation layer is non-brittle and can withstand the pressure of the test strip.

20. The carrier system of claim 18 where the red blood cell separation layer is impregnated with Poly Vinyl Alcohol.

21. A dry test strip having a plurality of layers, a first layer of the plurality of layers for filtering red blood cells, the first layer comprising:
a Borosilicate Glass Fiber layer; and
lectin, impregnated in the borosilicate layer, such that the dry test strip is configured to filter red blood cells from a blood sample.

22. A method of converting a hydrophobic filtering layer for use in red blood cell separation, the method comprising:
(a) providing a filtering layer;
(b) adding lectins causing the filtering layer to agglutinate RBCs.

23. The method of claim 22, wherein the filtering layer is a borosilicate glass layer.

24. The method of claim 23, further comprising:
(c) adding poly vinyl alcohol causing the filtering layer to be less brittle and improving its hydrophilic nature.

25. The method of claim 24, further comprising:
(d) adding an additive selected from the list consisting of Sorbitol/Mannitol and Sucralose/Sorbitol.
